SEQUENCE LISTING (10 sequences)

SEQ ID NO: 1 - DNA, 21 bp, Artificial Sequence
  Description of artificial sequence Primer
ttcaacagtctaagcggagtg

SEQ ID NO: 2 - DNA, 21 bp, Artificial Sequence
  Description of artificial sequence Primer
aaatgaattctatgtatgggg

SEQ ID NO: 3 - DNA, 90 bp, Artificial Sequence
  Description of artificial sequence sense primer P1
ggggcggccgcagaacaaaaactcatctcagaagaggatctgtctagataggacgatgacgataagactgttgaaagttgtttagcaaaa

SEQ ID NO: 4 - DNA, 23 bp, Artificial Sequence
  Description of artificial sequence antisense primer P2
acgaatggatcctcattaaagcc

SEQ ID NO: 5 - DNA, 21 bp, Artificial Sequence
  Description of artificial sequence sense primer P3
gattacgccaagctttggagc

SEQ ID NO: 6 - DNA, 63 bp, Artificial Sequence
  Description of artificial sequence antisense primer P4
ctcttctgagatgtgtttttgttcttggccacgtcggccacgtttgatttccaccttggtccc

SEQ ID NO: 7 - DNA, 63 bp, Artificial Sequence
  Description of artificial sequence sense primer P5
gaacaaaaactcatctcagaagaggatctgaaacgtgaagacgatgacgataagactgttgaa

SEQ ID NO: 8 - DNA, 13 bp, Artificial Sequence
  Description of artificial sequence Primer
ggcccagccggcc

SEQ ID NO: 9 - DNA, 13 bp, Artificial Sequence
  Description of artificial sequence Primer
ggccgacgtggcc

SEQ ID NO: 10 - DNA, 108 bp, Artificial Sequence
  Description of artificial sequence gIII 5′ end of mutant halper phage having 2 amber codon
gtgaaaaaattattattcgcaattcctttagttgttcctttctattctcactccgcttagactgttgaaagttgtttagcaaaaccccatacatagaattcatttact

(19) United States
(12) Patent Application Publication
(10) Pub. No.: US 2004/0266007 A1
(43) Pub. Date: Dec. 30, 2004

(54) Mutant helper phase for isolation of antibody molecules in phage display

(76) Inventor: Sang-Hoon Cha, Seoksa-dong (KR)

Correspondence Address: Jones Day, 1155 Avenue of the Americas, New York, NY, 10036, US

(21) Appl. No.: 10/488,428

(22) PCT Filed: May 28, 2002
(86) PCT No.: PCT/KR02/01001

(30) Foreign Application Priority Data
Aug. 29, 2001 (KR) 2001/52451

(51) Int. Cl. C12N 7/00; C12N 15/86

(52) U.S. Cl. 435/457; 435/235.1

(57) ABSTRACT

The present invention relates to a genetically modified helper phage, named Ex-phage, for packaging phagemid vector. For the modification, amber codons are introduced at 5′region of the helper phage genome by site-directed mutagenesis. The resulted mutant helper phage produces wild-type pIII in suppressive strains but not in non-suppressive strains. Furthermore, this invention provides a method of preparing phage display library expressing various foreign proteins on the surfaces of the phages.

### TECHNICAL FIELD

This invention relates to a mutant helper phage to increase display level of foreign polypeptides on the surface of recombinant phage in phage display technology, and the use of the mutant helper phage.

### BACKGROUND ART

Combinatorial library denotes a systemic collection of thousands of diverse molecules, where each of molecules is composed of 10 or more molecular units. Typical example of a combinatorial library is a phage-displayed peptide library composed of 10⁷ or 10⁸ different phage generated by modifying amino acid composition of a part of coat proteins of bacteriophage through genetic manipulation using molecular biological approaches (Cwirla et al., Proceedings of National Academy of Science USA, 87:6578, 1990).

Mixture of short peptides synthesized by different amino acid combination or a low molecular material generated by different combination of replacements on the branches of a main framework is the example of a combinatorial library. The feature of combinatorial library is that although it is composed of millions of different phage particles, high throughput screening to find specific particles having a new and physiological effect without inspecting each and every numerous phage is possible.

Thus, the importance of the library in the area of drug development is beginning to be appreciated recently.

Conventional procedure for obtaining a phage display library comprising the steps of: i) Introduction of any oligonucleotides at a site corresponding to the N-terminus of pIII (or pVIII) coat protein of phage; ii) Display of fusion proteins that have a part of wild type coat protein fused with polypeptides encoded by the introduced oligonucleotides; iii) Preparation of receptor molecules that bind to the polypeptides encoded by oligonucleotides; iv) Elution of (poly)peptide-phage particles by applying low pH or molecules with competitive binding activity thereto (biopanning); v) Amplification of eluted phage using host cells; vi) Repetition of above steps to enrich specific phage particles; vii) Determination of amino acid sequences of positive peptides by deducing from DNA sequence of selected phage clones obtained by panning.

In accordance with the procedure described above, EcOR I endonuclease was fused to the minor capsid protein pIII, thereby EcOR I-gIII fusion protein had been initially displayed on the surface of M13 virus particles. Thus, according to the conventional procedure, one can obtain a huge library expressing foreign proteins. So Far, a wide range of biomolecules, such as proteins or protein domains, have been displayed on the surface of phage for carrying out directed evolution of the molecules. For example, stronger binding ligands for a receptor, enzyme inhibitors, DNA binding proteins, antagonists, or antibodies specific for various antigens have been identified using a phage display technology.

In general, there are two types of vectors that have been used for the display of exogenous genes on the surface of filamentous phage. One is a phage vector (fUSE5, fAFF1, fd-CAT1 or fdtetDOG) and the other is a phagemid vector (pHEN1, pComb3, pComb8 or pSEX).

In a phage vector system, peptides can be displayed as gIII fusion for oligovalent expression (Scott J. K. and Smith G. P., Science 249: 386-390, 1990) or gVIII fusion for multivalent expressions (Greenwood J. et al., J. Mol. Biol. 220: 821-827,1991) by cloning synthesized genes directly within the phage genome. Thus, a phage vector system could provide a high display level of foreign peptides or protein fragments so long as all pIII molecules are originally presented as fusions without degradation. However, there is limitation in size of exogenous protein fragments fused with pIII (>100 amino acids) since the presence of a large foreign protein fragment at the N-terminal of pIII hinders the interaction of pIII with sex pili on bacterium that is absolutely required at the initial step of phage infection. In case of pVIII, fusion with amino acid residues bigger than 10 compromises coat protein function in general, although there has been recent publication demonstrated that much larger protein fragments could be display as pVIII fusions (Sidhu S. S., Curr. Op. Biotechnol. 11: 610-616, 2000).

For the display of larger molecules such as antibodies, therefore, a phagemid vector system is more suitable. In addition, a phagemid vector system has more advantages over a phage vector system including higher efficiency in ligation-transformation step which allows creating larger libraries and relatively easy genetic manipulation for introducing special features into a phagemid. In a phagemid vector system, DNA of exogenous proteins are cloned into gIII (or gVIII) within a phagemid vector, and the packaging of recombinant phagemid DNA and display of the fusions are provided by a helper phage such as M13KO7 or VCSM13. Thereby, the phagemid presents modified capsid proteins as fusions, and a helper phage supplies wild-type version of the coat proteins that is required for the successful reinfection of recombinant phage for amplification. The resulting phage particles display pIII from both wild-type pIII of the helper phage and the fusion pIII from the resident phagemid. In reality, however, the majority of pIII molecules displayed on the surfaces of phage particles are in wild-type pIII because of proteolytic degradation of the pIII:fusion protein at the periplasmic space of E. coli. This implies that a large proportion of phage particles is actually “bald” with respect to display of fusion in a phagemid display system, and the low display level results in low efficiency of isolating specific binding molecules from a library. In a phagemid display system, therefore, a new strategy to achieve high level display of pIII:fusion protein on the surfaces of recombinant phage is needed for the successful isolation of diverse specific binders from a phage display library.

To get around this problem, M13 helper phage with gIII deletion (M13δg3) (Griffith A. D. et al., EMBO J. 12: 725-734, 1993) had been designed to observe the enhancement of display level. However, the titer of the helper phage produced by using method as above is too low (about 10⁹/1) to satisfy the amount of helper phage required for the packaging. Recently, this strategy is slightly modified further. A packaging cell line (DH5α/pIII) was generated by inserting M13 gIII into the chromosome of DH5α cells, and high titer of hyperphage was produced by transformation of M13KO7ApIII helper phage DNA into DH5α/pIII cells (Rondot S. et al., Nat. Biotech. 19: 75-78, 2001). Mutation of the signal sequence and use of helper phage with trypsin-cleavable pIII coat protein also have been reported for improvement of the display of proteins on filamentous phage (Jestin J. et al., Res. Microbiol. 152: 187-191,2001).

Thus, it is an objective of this invention to provide a mutant helper phage for packaging a phagemid vector containing filamentous virus genome of which at least a part of gene of natural minor coat protein is deleted or defective. In the virus genome, conditional suppressive translation stop codon(s) is introduced into the N-terminus of the genome.

It is another objective of this invention to provide a phage display library expressing fusion proteins on the surface of the phage using the system.

### DISCLOSURE OF INVENTION

The present invention relates to the development of a mutant helper phage that increases the efficiency of specific antigen binding of recombinant phage particles in order to isolate specific and diverse antibody molecules to target antigens through phage display technology and the provision of a phage display library expressing foreign proteins with genetic diversity using the helper phage.

Thus, the present invention provides a helper phage for packaging a phagemid vector containing filamentous phage genome of which, at least, a part of the gene of wild type minor coat protein, or deleted or defective filamentous phage genome, wherein conditional suppressive translation stop codons are introduced at the N-terminal of the gene of minor coat protein of the mutant helper phage.

In addition, the present invention provides use and methods of constructing a phage display library that expresses diverse ligand-binding proteins using the mutant helper phage described above.

In this invention, “conditional suppressive” translation stop codon means that the codon terminates the translation of a protein in non-suppressive strains, but is translated to an appropriate amino acid resulting in synthesis of normal protein in suppressive strains.

The mutant helper phage or the phagemid packaged by the said mutant helper phage (described above) may contain whole or a part of the genome of filamentous phage. Examples of such filamentous phages include, but are not limited to, fd, M13, f1, If1, Ike, Zj/Z, Ff, Xf, Pf1, Pf3 and their derivatives. The preferred minor coat protein, which is fused with foreign proteins, is pIII protein of fd, M13, f1, If1, Ike, Zj/Z of Ff, or a correspondent of the pIII protein presented on XF, PF1 or Pf3.

Conditional suppressive translation stop codon included in the mutant helper phage is UAG (Amber), UAA (Ocher) or UGA (Opel) codon, and introduction of the codon is achieved by insertion or replacement of the codon at the N-terminal of the minor coat protein gene. It is preferred to introduce two or more conditional suppressive translation stop codons at the N-terminal of the minor coat protein gene. For example, substitution as a translation stop codon can be achieved by replacing a codon for glutamic acid at the end of N-terminal of a minor coat protein gene to UAG (Amber) codon. For this experimental procedure, it is desirable to use the gene for N-terminal minor coat protein within the size of 90 amino acids containing pIII leader sequence.

As above, use of well-known phage, such as M13KO7, M13R408, M13-VCS or PhiX174, is desired for the introduction of translation stop codons for packaging of a phagemid vector, but not exclusively.

In this invention, the experimental examples showed that the backbone of a helper phage for the package of a phagemid vector was M13KO7, minor coat protein was pIII, and the mutant helper phage containing substitutions of 20th and 32th glutamic acids at the N-terminal of pIII with UAG codons was provided. The present mutant helper phage, named Ex-phage, has a genome with gIII containing two amber codons at its 5′ end whose DNA sequence is written in SEQ. ID No.: 10 (FIG. 1), and was deposited at the Gene bank of Korea Research Institute of Bioscience and Biotechnology at Jul. 24, 2001 (Deposit number: KCTC 10022BP).

The mutant M13KO7 with an amber codon at the first Glu residue in the N-terminal of mature pIII still formed plaques in non-suppressing JS5 cells, and those with an amber codon at the end of signal peptide or the second Glu position showed a few revertants. The Ex-phage produced clear plaque in suppressing host cell(s), such as TG1 and/or XL-1 blue cell (SupE genotype), but not in non-suppressing host cell(s) such as JS5 and/or MV1184 cell at all (FIG. 2). These results demonstrated that Ex-phage can propagate in suppressing E. coli strains since amber codons are translated for Glu but not in non-suppressing E. coli strains because of premature stop of translation by two nonsense codons.

In addition, this invention provides the methods for the generation of a phage display library that expresses diverse ligand-binding proteins on the surfaces using the mutant helper phage and the use of the library described above.

Methods for the generation of a phage display library described above contain following steps:

i) Generation of a recombinant phagemid that expresses an active foreign protein as fused with anchor domain of pIII.

ii) Coinfection of the phagemid in “step i)” and our invented mutant helper phage (Ex-phage) into non-suppressive host cells.

iii) Production of recombinant virus expressing at least one or more pIII-heterogeneous fusion proteins among its minor coat pIII proteins by growing host cells above.

The phagemid in “step i)” preferably contains genome of filamentous phage such as fd, M13, f1, If1, Ike, Zj/Z, Ff, Xf, Pfl or Pf3, but not limited thereto.

In “step i)” active heterogeneous proteins that are expressed as fusions with pIII anchor domains are mammalian proteins such as immunoglobulins or ligand-binding proteins. For examples, growth hormone, human growth hormone, des-N-methionyl growth hormone, bovine growth hormone, parathyroid hormone, thyroxine, insulin A-chain, insulin B-chain, proinsulin, relaxin A-chain, relaxin B-chain, prorelaxin, follicle stimulating hormone (FSH), thyroid stimulating hormone (TSH), luteinizing hormone (LH), glycoprotein hormone recepter, calcitonin, glucagon, factor VII, lung surfactant, urokinase, streptokinase, human tissue-type plasminogen activator, bombesin, factor IX, thrombin, hemopoietic growth factor, tumor necrosis factor-α and -β, enkephalinase, human serum albumin, mullerian-inhibiting substance, mouse gonadotropin-associated peptide, β-lactamase, tissue factor protein, inhibin, activin, vascular endothelial growth factor, integrin receptor, thrombopoietin, protein A and D, rheumatoid factor, nerve growth factor, platelet growth factor, transforming growth factor (TGF), TGF-α and TGF-β, insulin-like growth factor I and II, insulin-like growth factor binding protein, CD-4, DNase, latency associated peptide, erythropoietin, heregulin 2 factor (HER2), osteoinductive factor, interferon-α, β and γ, colony stimulating factor (CSFs), M-CSF, GM-CSF, G-CSF, interleukin (ILs), IL-1, IL-2, IL-3, IL-4, superoxide dismutase, decay-stimulating factor, viral antigen, HIV envelop protein, GP120, GP140, atrial natriuretic peptide A, B and C, immunoglobulin or fragments of proteins described above can be used, but not exclusive.

In one embodiment of the invention, human immunoglobulin was used as a heterogeneous protein. In this case, human immunoglobulin is preferred to contain heavy chain variable domains and light chain variable domains, and use of scFv (single chain Fv) in which a heavy chain variable domain and a light chain variable domain of human immunoglobulin is connected each other by a linker is desired.

In addition, the recombinant phagemid of “step i)” could express a fusion protein containing an enterokinase cleavage site between pIII anchor domain and active heterogeneous protein.

In another embodiment of the invention, a recombinant phagemid, named pIGT3, shown in FIG. 3B was constructed, and was deposited at the Gene bank of Korea Research Institute of Bioscience and Biotechnology at Jun. 24, 2001 (Deposit number: KCTC10021BP).

The said phagemid vector produced antibody fusion proteins which was fused with pIII and contained trypsin and enterokinase cleavage sites for proteolytic elution of phage.

In these examples, Ex-phage had a mutant pIII gene that produced a functional wild type pIII in suppressive E. coli strains but did not make any pIII in non-suppressive E. coli strains. Packaging the said phagemids encoding antibody-pIII fusion in F+non-suppressive E. coli strains with Ex-phage enhanced the display level of antibody fragments on the surfaces of recombinant phage particles.

In “step ii)”, non-suppressive E. coli strains such as MV1184, MV1193, XS101, XS127 or JS5 cell can be used as a host cell, and it is preferred for the strain to be coinfected with the recombinant phagemid and the mutant helper phage in ratio of 1:10 to 1:20 in order to produce high titer of recombinant virus.

In between “step i)” and “step ii)”, additional step can be included for the mass production of the mutant helper phage by infecting the mutant helper phage into host cells. Suppressive E. coli strain such as DH5α F′, JM101, JM109, JM110, KK2186, TG1 or XL-1 Blue cell can be used as a host cell.

A phage display library generated by the methods above can be screened for recombinant virus expressing specific antibodies binding to target antigens through affinity selection in order to produce antibody molecules that bind to specific antigens. Antibody molecules expressed on the selected recombinant virus can be easily purified by proteolytic elution with trypsin or enterokinase.

The phage display library system in the present invention has following features: i) Use of mutant helper phage expressing genetically modified gIII containing not less than two conditional suppressive translation stop codons; ii) Construction of a phage display library in non-suppressive E. coli strains that have been used for the production of soluble antibody molecules previously; iii) Having advantage of going around technical complications caused by trypsin elution during panning by using enterokinase which is more specific protease than trypsin. Therefore, a phage display library in the present invention can be used effectively at probing candidate molecules for the development of therapeutic antibody drugs by screening diverse antibodies specific for target antigens.

### BRIEF DESCRIPTION OF DRAWINGS

FIG. 1 shows genome of Ex-phage generated by site-directed mutagenesis of M13KO7 helper phage genome.

FIG. 2 shows plaque formation of Ex-phage on suppressive E. coli. Phage solution containing the same Ex-phage clone was spotted on top agar with suppressive or nonsuppressive E. coli strains and incubated.

FIG. 3A illustrates the construction of pIGT2 and pIGT3.

FIG. 3B illustrates detailed diagram of pIGT3.

FIG. 4 shows the Diagram of Ex-phage system.

FIG. 5 illustrates the determination of scFV:pIII fusion protein expression by immunoblot.

Lane 1: recombinant phage particles obtained by infecting with M13KO7 helper phage (pIGT3/M13KO7).

Lane 2: recombinant phage particles obtained by infecting with Ex-phage (pIGT3/Ex-phage)

FIG. 6A shows antigen binding specificity of recombinant phage packaged with either M13KO7 or Ex-phage.

FIG. 6B shows antigen binding sensitivity of phage particles packaged with either M13KO7 or Ex-phage.

FIG. 7 shows enrichment of panning efficiency by Ex-phage package pIGT3/M13KO7 or pIGT3/Ex-phage FIG. 7A shows percentage yield after panning.

FIG. 7B shows polyclonal phage ELISA on human HSP-70 protein after panning.

FIG. 7C shows monoclonal phage ELISA on human HSP-70 protein.

### BEST MODE FOR CARRYING OUT THE INVENTION

Herein after, the present invention is explained in detail based on experimental results.

Any publications referenced herein are hereby incorporated by reference in this application in order to more fully describe the state of the art to which the present invention pertains.

It is important to an understanding of the present invention to note that all technical and scientific terms used herein, unless otherwise defined, are intended to have the same meaning as commonly understood by one of ordinary skill in the art. The techniques employed herein are also those that are known to one of ordinary skill in the art, unless stated otherwise.

Reference to particular buffers, media, reagents, cells culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

### EXAMPLE 1

### Mutagenesis of M13KO7 Helper Phage Genome

Amber codon (TAG) was introduced at the 5′ region of gIII of M13KO7 helper phage genome (Stratagene, USA) by site-directed mutagenesis (Kunkel, T. A., Proc. Acad. Sci. USA 82: 488-491, 1985) using Mutan™-K enzyme and vector set (Takara, Japan).

More specifically, single strand DNA of M13KO7 helper phage including deoxyuridine was prepared by infecting CJ236 indicator cell with the phage, wherein the cell was lacking of dUTPase and Uracil-N glycosylase.

A single plaque of M13KO7 was inoculated into 3 ml of 2×YT medium (30 μg/ml chloramphenicol) containing CJ236 indicator cell and incubated at 37° C. for 6 hrs. After spin the culture, added 100 ml of supernatant to 100 ml of 2×YT medium (30 μg/ml chloramphenicol) containing CJ236 cells and incubated at 37° C. for 6 hrs. After spin the culture, amplified phage was precipitated by adding ¼ volume of PEG/NaCl solution to the supernatant. Phage pellet was resuspended in 5 ml of TE buffer and single strand DNA was purified by phenol/chloroform extraction.

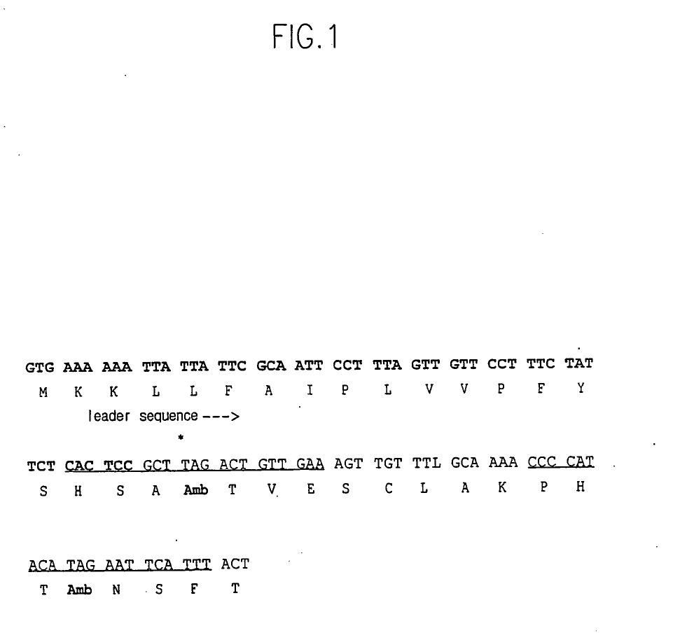

Two oligonucleotides (SEQ. ID No.:1 and SEQ. ID No.: 2) were synthesized for the mutagenesis. Location of complementary sequences to the oligonucleotides used in the experiment at the gIII was shown in FIG. 1. 10 pmole of oligonucleotide (SEQ. ID No.:1) was phosphorylated with 10 units of T4 polynucleotide kinase (from Roche) at 37° C. for 15 min. Complementary strand was synthesized by treating E. coli ligase (60 units) and T4 DNA polymerase (1 unit) at 25° C. for 2 h followed by adding 0.2 pmol of single strand phage DNA and 0.1 pmol of phosphorylated oligonucleotide. Then, 10 microliters of the DNA were mixed with 100 microliters of BMH71-18mutS competent cell (from Takara). The mixture was heated up to 42° C. and shaked for 45 seconds for the infection of cells. The infected cells were cultured in 1 ml of LB-medium at 37° C. for 1 hr. Serial dilutions of cells (x10⁻¹, x10⁻², and x10⁻³) were prepared. After mixing the respective dilutions with XL-1 Blue cells (Stratagene), 3 ml of top agar was added to the mixture. The mixture was relocated onto LB plate and incubated at 37° C. overnight to form plaques.

Any one of the plaques was selected randomly. Phages are released on 500 microliters of LB at room temperature for 2 h. 2 microliters of phage suspension was added to top agar LB medium containing TG1 (American Pharmaceuticals) or JS5 (Biorad) bacterial lawn (Biorad), then cultured at plate at 37° C. overnight. The mutant helper phage were identified by comparison of plaque formation on TG1 (suppressive strain) or JS5 cells (non-suppressive strain). Then the mutant phage that form clear plaques on TG1 cells but not on JS-5 cells were isolated, single-stranded DNA was purified from the isolated phage, and the second round of site-directed mutagenesis was performed using SEQ. ID No.:2.

The resulting mutant phage, named Ex-phage, has two amber codons at the 5′ region of gIII (Deposit number: KCTC10022BP).

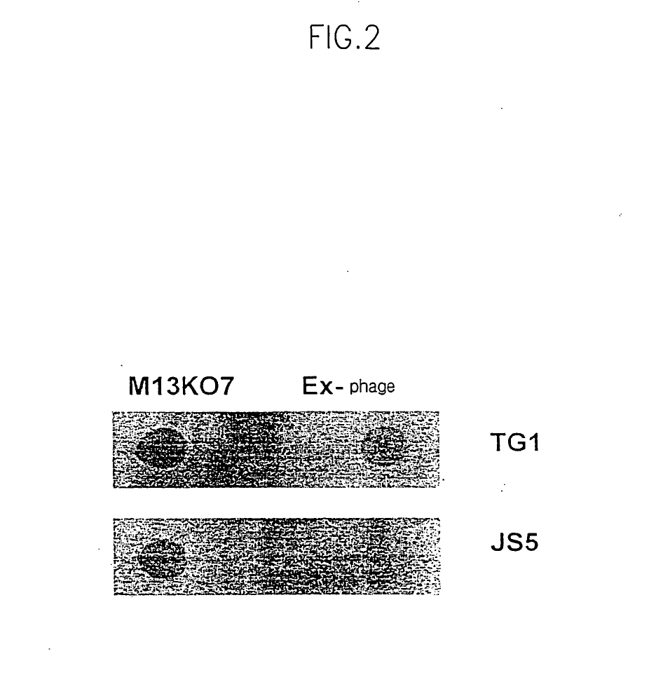

The Ex-phage produced clear plaque in TG1 cells (supE genotype) but not in JS5 cells at all (FIG. 2).

### EXAMPLE 2

### Construction of Phagemid Vectors

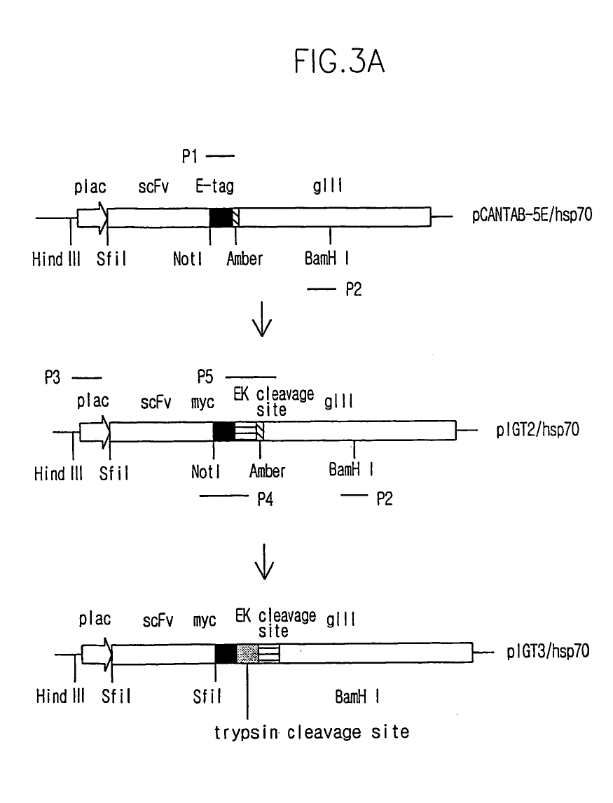

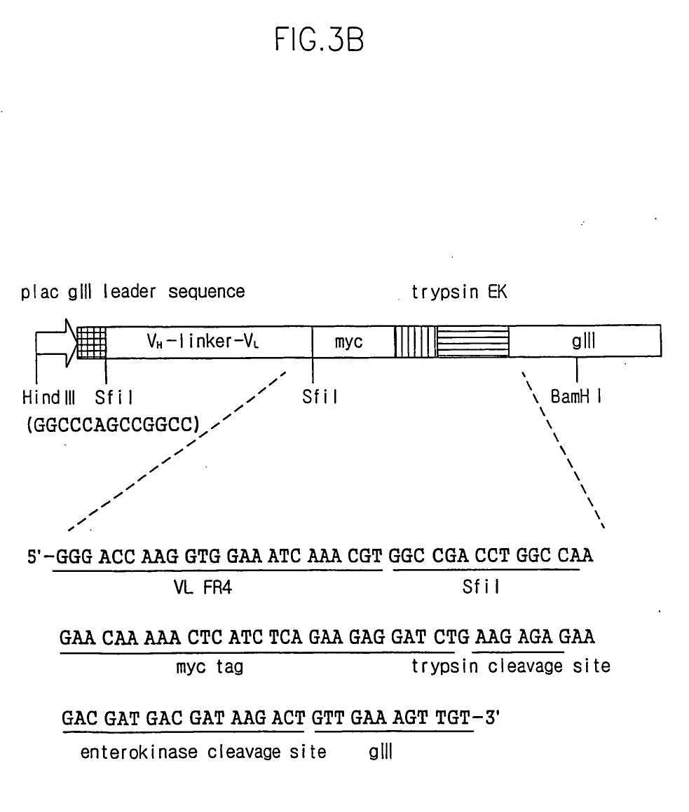

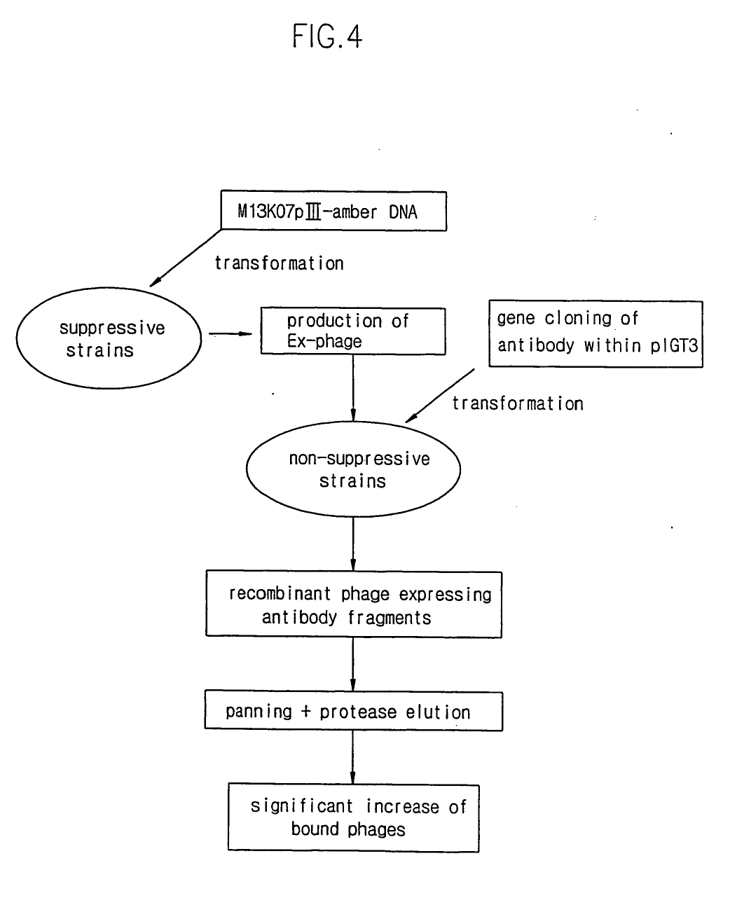

In order to apply the Ex-phage obtained from the example 1 in phage display, pIGT2 and finally pIGT3 phagemid vectors were constructed by genetic modification of pCANTAB-5E, yet pUC119 backbone of pCANTAB-5E was not altered (FIGS. 3A and 3B).

2-1) Construction of pCANTAB-5E/hsp70

The pCANTAB-5E/hsp70, specific for human recombinant HSP-70 (heat shock protein 70) was isolated from a semi-synthetic scFv library by panning method.

Peripheral blood lymphocytes (PBL) were obtained from 40 healthy volunteers. Total RNA was isolated from these cells using RNA STAT-60 (TE-TEST), and 1^{st} strand cDNA was synthesized with 1^{st} strand cDNA synthesis kit (Roche Biochemicals, Germany) for PCR template. In addition, lambda DNA was purified from the human bone marrow (BM) 5′-STRETCH PLUS cDNA library and human fatal liver (FL) 5′-STRETCH PLUS cDNA library (Clonetech, USA), and was also used as a template to amplify the human scFv gene fragments. Linker fragment that joins V_{H} and V_{L} domains was obtained from a scFv gene fragment in pHEN1 (kindly provided by Dr. Greg Winter in Cambridge Antibody Technologies, Ltd., Daly Research Laboratories) using human linker specific primers (sense primer: 5′-GRACMMYGGTCACCGTCTCYTCAGGTGG-3′, antisense primer: 5′-GGAGACTGNGTCAWCWSRAYDTCCGATCCGCC-3′, which were made by Bioneer Co., Korea). The resulting V_{H}, V_{L} and linker fragments were purified with (1%) low melting agarose gel and quantified. Full length scFv genes (about 750 bp) were obtained by a series of assembly PCR and pull-through PCR amplifications, purified using low melting agarose gel, and digested using Sfi I and Not I restriction enzymes. PCANTAB-5E vector was digested with the same restriction enzymes and treated with CIP (Calf Intestinal Alkaline Phosphatase, Roche). Obtained scFv gene fragment and PCANTAB-5E vector were ligated using T4 DNA ligase (Promega). The resulting ligated reaction was used to transform TG1 ultra-competent cells. The resulting library size was 5×10⁸.

The library was inoculated into a medium (2×YT/AG; 100 μg/ml ampicillin, 2% Glucose) and was incubated to amplify recombinant phages.

Recombinant phages were amplified using M13KO7 helper phage. Panning was performed adding 10¹² of amplified recombinant phage particles. Briefly, a 96-well plate was coated with 50 μg/ml of human recombinant HSP-70 in coating buffer (0.1 M NaHCO₃, pH 9.6) overnight at 4° C., and blocked with 3% bovine serum albumin (BSA) (Sigma Co.) for 1 h at 37° C. Then, total 1012 recombinant phage were added, and incubated for 2 h at room temperature (RT). The 96-well was washed with PBS containing 0.1% tween-20 (polyoxyethylene sorbitan monolate) (PBS-tween) Bound phage were eluted with elution buffer (0.1 M HCl). The titer of eluted phage was determined. The eluted phage were amplified by infecting freshly grown TG1 cells. Panning was repeated 4 times.

The yield was increased 100-fold in 2^{nd} and 1000-fold in 4^{th} panning. The yield of 3^{rd} was decreased 10-fold compared to 2^{nd}, but increased 10-fold compared to 1^{st} round. Enrichment of antigen-specific phage was determined by polyclonal phage ELISA by adding about 10¹² phage particles. The result of polyclonal phage ELISA showed that the presence of positive phage was increased in the 3^{rd} and the 4^{th} rounds of panning.

In order to identify positive phage clones, monoclonal phage ELISA was performed using culture supernatant containing monoclonal phage particles that obtained by overnight culture of random 100 colonies at the 3^{rd} round of panning. A 96-well plate was coated with recombinant HSP-70.1 or BSA was blocked. Anti-M13 tag antibody conjugated with horse radish peroxidase (HRPO) (Amersham Pharmacia Biotech) was added into each well. The plate was analyzed at O.D. 405 nm. About 15 clones out of 100 colonies showed high absorbance more than 0.5. Most of the colonies showed significantly high reactivity to HSP-70.1. One clone showing the highest binding signal with hsp-70 protein was selected and named it pCANTAB5E/HSP70.

2-2) Construction of pIGT2

In pIGT2, E-tag of pCANTAB-5E was replaced with myc tag and an EK cleavage site was introduced into the vector. More specifically, 600 bp of gIII fragments between Not I and BamHI sites in pCANTAB-5E were obtained by PCR amplification. The sense primer P1 (SEQ. ID No.:3) was designed to contain Not I restriction enzyme site, myc tag, Xba I restriction enzyme site, an amber codon, EK cleavage site and the sequence complementary to 5′ region of gIII. Antisense primer P2 (SEQ. ID No.:4) was complementary to the middle of gIII region with BamH I restirction enzyme site.

The resulting 600 bp PCR product was treated with Not I/BamH I, and purified with Wizard DNA clean up kit (Promega, USA). The pCANTAB/hsp70 was restricted with the same set of restriction enzymes and purified with 1% low melting temperature agarose gel for eliminating the original 600 bp of Not I/BamH I DNA fragment including E-tag sequence. The resulting vector fragment and the PCR product were ligated together using T4 DNA ligase (Promega) at 16° C. overnight, and transformed into HB2151(Amersham Pharmacia) electrocompetent cells. Bacterial colonies were randomly picked after incubating cells on 2×YT/Amp plate (100 μg/in ampicillin) at 37° C. overnight, and grown in LB/Amp plate in the presence of 1 mM isopropyl-β-D-thiogalactoside (IPTG) (Sigma Co. USA). Total cellular proteins were separated with 12% SDS-PAGE, and transferred to nitrocellulose membrane (Biorad). The clones expressing a human anti-hsp-70 scFv fused with myc tag at its C-terminal were identified by immunoblot using the 9E10 anti-myc mAb (ATCC, USA) (Evan G. I. et al., Mol. Cell. Biol. 5: 3610-3616, 1985).

2-3) Construction of pIGT3

pIGT3 was generated by replacing Not I restriction enzyme site of pIGT2 with Sfi I, and introducing trypsin cleavage sequence between myc tag and EK cleavage site of pIGT2. Moreover, an amber codon in front of gIII was removed overlapping PCR was carried out for those modifications and pIGT2 was used as a PCR template as shown in FIG. 3A. The first PCR fragment (800 bp) for replacing Not I cloning site of pIGT2 with Sfi I was obtained by using a sense primer P3 (SEQ. ID No.:5) and an antisense primer P4 (SEQ. ID No.:6). The PCR for obtaining the first fragment was repeated 25 times (at 94° C. for 1 min., at 62° C. 1 min., and at 72° C. 1 min.) The second PCR fragment (600 bp) for introducing trypsin cleavage sequence and removing an amber codon of pIGT2 was obtained by using a sense primer P5 (SEQ. ID No.:7) and an antisense primer P2 (SEQ. ID No.:4) The PCR for obtaining the second fragment was repeated 25 times (at 84° C. for 1 min., at 62° C. for 1 min., and at 72° C. for 1 min.) The resulting two PCR fragments were linked together by overlapping PCR using mixture of 80 ng of each 1st PCR product and 2nd PCR product as templates and primers P3 (SEQ. ID No.:5) and P2 (SEQ. ID No.:4). This overlapping PCR was repeated 25 times (at 94° C. for 1 min., at 60° C. for 1 min., and at 72° C. for 1 min.). The resulting 1,400 bp PCR fragment was treated with Hind III/BamH I and cloned into pIGT2 in order to generate pIGT3 by substituting the original Hind III/BamH I fragment of pIGT2.

Recombinant phagemid pIGT3 in this invention was deposited at the Gene bank of Korea Research Institute of Bioscience and Biotechnology at Jun. 24, 2001 (Deposit number: KCTC10021BP).

### EXAMPLE 3

### Phage Quantification by Elisa

PIGT3 containing anti-hsp70 scFv-pIII fusion protein (pIGT3/hsp70) in JS5 cells were packaged with either Ex-phage (pIGT3/Ex-phage) or M13KO7 helper phage (pIGT3/M13KO7), and the assembly of functional phage antibody particles was monitored. Yields of pIGT3/Ex-phage and pIGT3/M13KO7 were determined by phage ELISA.

JS5 cells carrying pIGT3 encoding anti-hsp70 scFv:pIII fusion protein (pIGT3/hsp70) were infected with either M13KO7 helper phage or Ex-phage preparation at an OD₆₀₀ of 0.5 at a multiplicity of infection (M.O.I.) of 20 for 1 h. Then spin the culture and the pellet was resuspended in fresh 2×YT/AP (100 μg/ml ampicillin and 1 mM IPTG) and cultured at 30° C. overnight. Recombinant phage were precipitated using 25% PEG/NaCl.

Serial dilutions of phage in coating buffer (0.1 M NaHCO₃, pH 8.6) were coated in microtiter plates at 4° C. overnight. After blocking the plate with 1% bovine serum albumin (BSA) (Sigma Co.) in PBS (137 mM NaCl, 3 mM KCl, 8 mM Na₂HPO₄, 1 mM KH₂PO₄, pH 7.3), the bound phage were detected with anti-M13 antibody conjugated with horse radish peroxidase (HRPO) (Amersham Pharmacia). The signal was visualized with 2,2′-azino-di-(3-ethylbenzthiazoline-6-sulfonic acid) (ABTS) substrate and quantitated with ELISA reader (Biorad). M13KO7 helper phage of known plaque forming units (pfu) were used for standardization (Rondot S. et al., Nat. Biotech. 19: 75-78, 2001).

The number of phage particles produced by packaging with Ex-phage was the same with phage preparations using M13KO7 helper phage. And this result indicated that Ex-phage effectively used for the packaging of the function phage particles. (This indicated that the mutant helper phage in this invention was efficiently utilized for the assembly of functional phage particles.)

### EXAMPLE 4

### Immunoblot Analysis

To access potential effect of Ex-phage packaging on an increase of display level of the pIGT3 phage particles, the same number of pIGT3/Ex-phage and pIGT3/M13KO7 were analyzed by immunoblot using mouse mAb specific for pIII of M13.

High titers of M13KO7 helper phage and Ex-phage were prepared by infecting TG1 cells (Amersham Pharmacia) in 100 ml LB and incubated at 37° C. for 6 h with vigorous agitation in a shaking incubator. To obtain recombinant phage, a human anti-hsp70 scFv gene that obtained in our laboratory previously from a scFv phage display library constructed by using pCANTAB-5E vector was cloned into pIGT3, and JS5 cells carrying pIGT3-hsp70 phagemid were infected with either M13KO7 helper phage or Ex-phage preparation at a OD₆₀₀ of 0.5 at a multiplicity of infection of 10˜20 for 2 h in 50 ml LB containing ampicilin. Final concentration of 1 mM IPTG and 50 μg/ml kanamycin were added, and cultured at 30° C. overnight. The recombinant phage were harvested by centrifugation and purified by PEG precipitation. Phage proteins were separated by loading approximately 10¹⁰ recombinant phage into each lane of 10% SDS-PAGE, and transferred to nitrocellulose membrane (Amersham Pharmacia). The membrane was blocked with 3% skimmed milk solution in PBS for 1 h at room temperature. Immunoblot was carried on with anti-gIII monoclonal antibody (mAb) (Mobitec, Germany), and goat anti-mouse IgG antibody conjugated with HRPO (Sigma Co.) was used for the secondary antibody. The signal was visualized on X-ray film (Roche, Germany) using ECL substrate (Amersham Pharmacia).

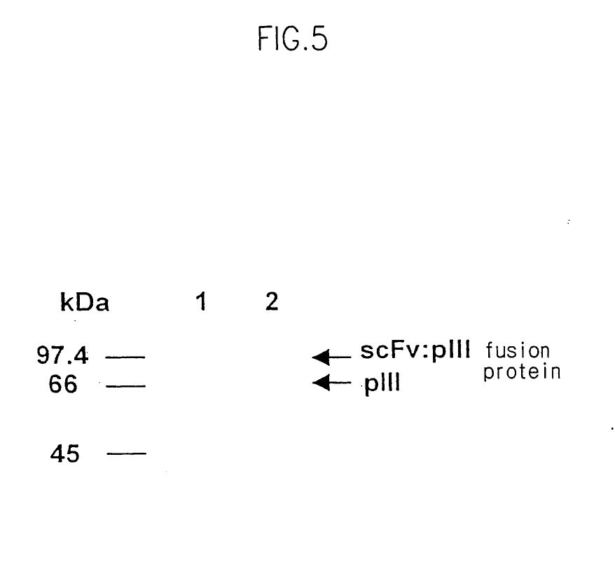

As expected, scFv:pIII fusion protein was the major form of pIII in pIGT3/Ex-phage, whereas most of pIII was wild-type in pIGT3/M13KO7 demonstrating dramatic increase of display level by Ex-phage (FIG. 5). Densitometry analysis of the immunoblot showed that only 5% of pIII were scFv:pIII fusion forms in pIGT3/M13KO7 indicating that only one out of four phage displayed only one scFv molecule on its surface, but three to four out of five pIII minor coat proteins were displayed as the scFv:pIII fusions on the surface of every pIGT3/Ex-phage.

### EXAMPLE 5

### Determination of Antigen-Binding Reactivity by Phage Elisa

5-1) Determination of Antigen-Binding Specificity

To determine antigen-binding specificity of recombinant phage particles, 100 ng of BSA, lysozyme (Sigma Co.), recombinant glutathione S-transferase (GST) or recombinant human HSP-70 in coating buffer (0.1 M NaHCO₃, pH 8.6) were coated in microtiter plates (Falcon) at 4° C. overnight. Recombinant GST protein was produced by growing DH-5α cells with PGEX vector (Amersham Pharmacia) in the presence of 1 mM IPTG, and affinity-purified by using glutathione agarose beads (Sigma Co.) Recombinant human HSP-70 protein was produced by growing BL21 (DE3) cells harboring pET28 vector (Invitrogen, USA) with human hsp-70 cDNA insert, and affinity-purified with Probond resin (Invitrogen). After blocking the plate with 1% BSA in PBS, 10¹⁰ scFv phage packaged with either M13KO7 or Ex-phage in 1% BSA solution were applied to each well for 1 h at room temperature. 10¹⁰ of M13KO7 helper phage were used as negative control. After washing 4 times with PBS containing 0.1% tween 20 (PBS-tween), the bound phage were detected with anti-M13 antibody conjugated with HRPO. The signal was visualized with ABTS substrate and quantitated with ELISA reader (Biorad) at OD₄₀₅.

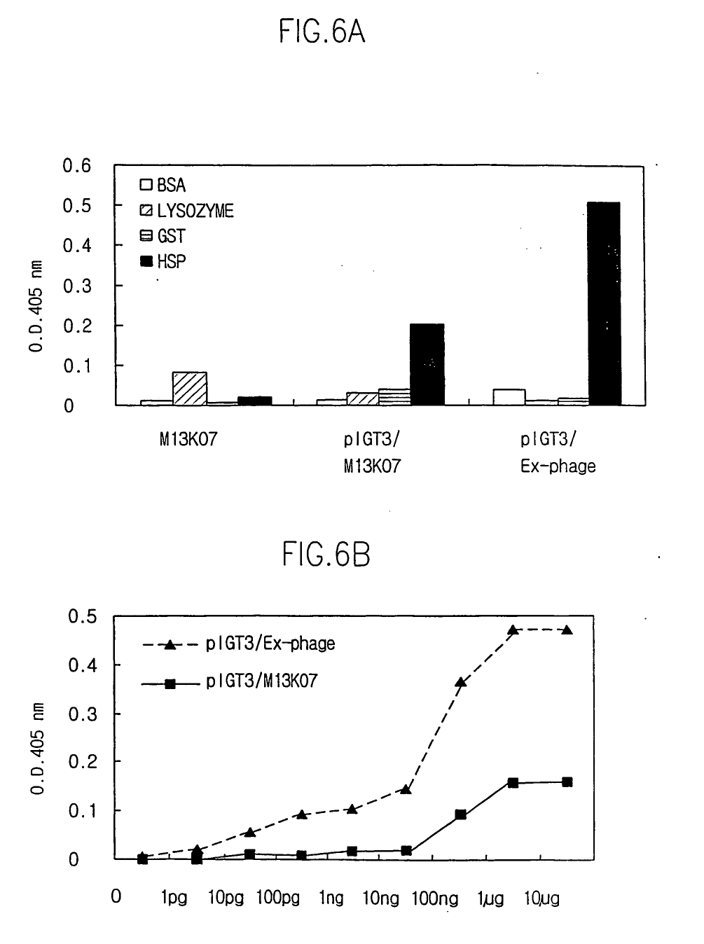

As shown in FIG. 6A, pIGT3/Ex-phage and pIGT3/M13KO7 phage particles specifically reacted with human recombinant HSP-70 protein only, but not to BSA, lysozyme or recombinant GST protein. In addition, pIGT3/Ex-phage gave not less than two times higher signal to the HSP-70 protein compared to pIGT3/M13KO7 indicating that an increase of display level enhanced antigen-binding signal through avidity effect.

5-2) Determination of Antigen-Binding Sensitivity

In order to determine antigen-binding sensitivity of recombinant phage, different concentrations of human recombinant HSP-70 protein (0 to 10 micrograms) were coated onto microtiter plates, and 10¹⁰ of pIGT3/Ex-phage or pIGT3/M13KO7 were tested for antigen-binding reactivity by phage ELISA.

pIGT3/Ex-phage bound at 100-fold lower concentration of the antigen compared with pIGT3/M13KO7 at the same ELISA signal, and gave positive signals at much smaller amounts of the antigen indicating that increase of displaying scFv:pIII fusion by Ex-phage directly enhanced antigen-binding sensitivity of recombinant phage particles (FIG. 6A).

### EXAMPLE 6

### Panning Procedure

To demonstrate the potential of Ex-phage for the efficient selection of specific binders over the large proportion of non-specific binders, pIGT3/M13KO7 and pIGT3/Ex-phage were mixed with abundant number of M13KO7 helper phage at 1:104, 1:106 or 1:108 dilution ratio, and two rounds of panning were carried out.

100 ng of recombinant human HSP-70 protein in coating buffer (0.1 M NaHCO₃, pH 9.6) was coated in microtiter plates at 4° C. overnight. Recombinant phage obtained by packaging pIGT3-hsp70 with either M13KO7 or Ex-phage were mixed with M13KO7 helper phage for non-specific background at 1:104, 1:106 or 1:108 ratio. After blocking the plate with 3% BSA in PBS, total 10¹⁰ phage from each diluting mixture were added into each well and incubated for 2 h at room temperature. Unbound phage were removed by washing with PBS-tween for six times with vigorous pipetting (chae J. et al., Mol. Cells 11: 7-12, 2001), and bound phage were eluted by 1 μg/ml trypsin treatment (Sigma Co.) (Rondot S. et al., Nat. Biotech. 19: 75-78, 2001).

Panning was repeated twice. The number of eluted phage was calculated by colony forming units (cfu) on JS5 cells in LB plates containing 50 μg/ml ampicillin (LB/amp plate) [Sambrook J. et al., Molecular cloning, A laboratory manual Edn. 2. (Cold Spring Harbor Laboratory Press, Cold Spring Harbor, N.Y.), 1989], and amplified phage were quantified by phage ELISA using M13KO7 helper phage as standards as described. Eluted phage from the first and the second round of panning were amplified in 50 ml of JS5 cells with either M13KO7 or Ex-phage superinfection and purified by PEG/NaCl precipitation. To determine the enrichment of antigen-specific phage after each round of panning, phage ELISA was performed with microplates coated with recombinant human HSP-70 protein.

For polyclonal phage ELISA, JS5 cell were infected with eluted phage for 20 min. and infected with either M13KO7 or Ex-phage preparation at an OD₆₀₀ of 0.5 at a M.O.I. of 10˜20 for 2 h in 50 ml of LB/Amp. Final concentration of 1 mM IPTG and 50 μg/ml kanamycin were added, and harvested by centrifugation and purified by PEG precipitation. 10¹⁰ of phage particles were added into microtiter plate, and performed phage ELISA. Phage ELISA using amplified phage particles after panning indicated that the panning selectively enriched the phage particles specifically bound to target antigen.

For monoclonal phage ELISA, JS5 cells were infected with eluted phage for 20 min. at room temperature, and spread onto LB/amp plates (100 μg/ml ampicillin). After overnight incubation at 37° C., E. coli colonies were randomly picked and inoculated into 200 ml LB/amp in sterile 96-well plates (Corning, USA). Individual phage clones were obtained by superinfecting E. coli clones with either M13KO7 or Ex-phage at 30° C. overnight. One hundred microliters of culture supernatant containing phage particles from the 96-well plates were added onto microtiter plates, and phage ELISA was performed as described. Through monoclonal ELISA recombinant phage displaying antigen-specific antibody were obtained and the frequency was determined. BSA was used as a negative control antigen and M13KO7 helper phage was used as a negative control phage.

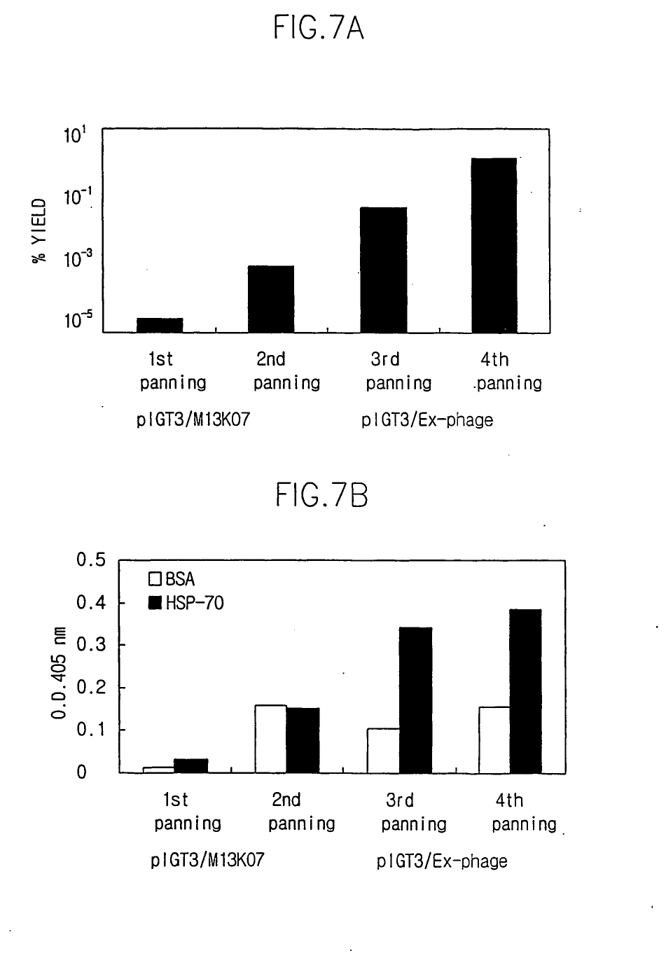

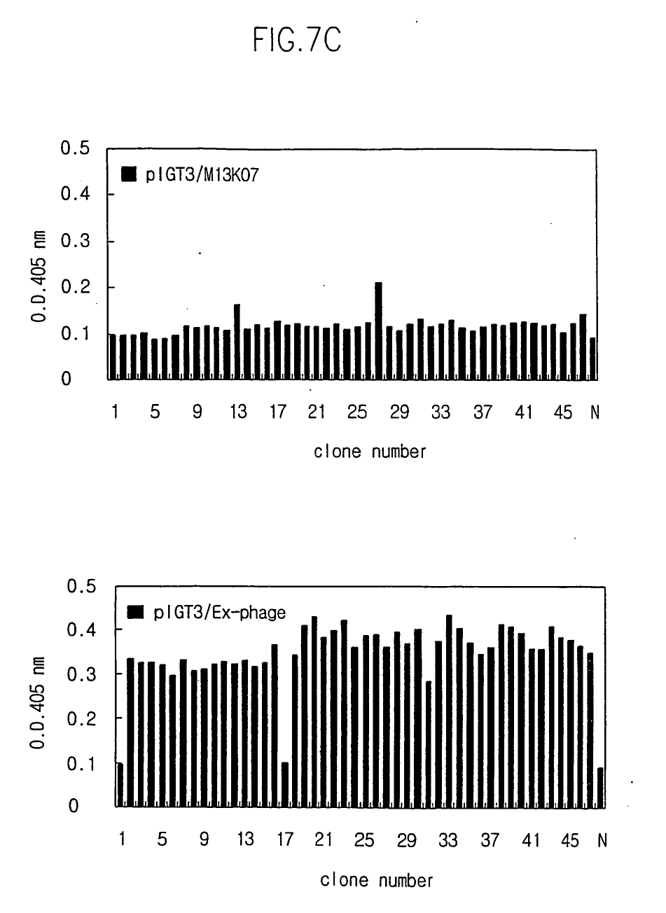

Both pIGT3/M13KO7 and pIGT3/Ex-phage did not show any enrichment at 1:108 dilution. Enhanced enrichment of antigen-specific phage was clearly observed in pIGT3/Ex-phage at 1:106 dilution (FIGS. 7A, B and C). FIG. 7A shows the percentage yield after each round of panning. Percentage yields after the second panning of pIGT3/M13KO7 and pIGT3/Ex-phage were increased 100-fold from the first panning, suggesting that the selective enrichment might occur during two consecutive panning by both pIGT3/M13KO7 and pIGT3/Ex-phage. PIGT3/Ex-phage gave about 10,000 times higher percentage yield compared to pIGT3/M13KO7, probably due to the higher binding reactivity to the antigen (FIG. 7A).

However, phage ELISA using amplified phage particles after panning indicated that an increase in percentage yield shown by pIGT3/M13KO7 was caused by non-specific binders, and only pIGT3/Ex-phage were selectively enriched among high background of M13KO7 helper phage by panning (FIG. 7B).

This was clearly proved by the monoclonal phage ELISA in FIG. 7C. 45 phage clones were randomly obtained by packaging with either M13KO7 helper phage or Ex-phage. Among 45 pIGT3/M13KO7 phage clones, only two clones were positive to a human hsp-70 protein. On the other hand, 43/45 pIGT3/Ex-phage clones gave a specific binding signal to a human hsp-70 demonstrating that >95% of pIGT3/Ex-phage after the second panning were positive.

So far, the present invention was described in detail based upon experimental results, but this invention is not restricted to the specific data presented above. Someone who has common knowledge in the research area relating to the present invention may understand that many modifications and changes can be added without getting out of the principle of the present invention.

### INDUSTRIAL APPLICABILITY

The use of the present mutant helper phage restored the display level of the fusions to that achieved from phage vectors. In our system, the Ex-phage can propagate in suppressive E. coli strains just like conventional M13KO7 helper phage. Packaging of recombinant phage was carried out in non-suppressive E. coli strains so that Ex-phage do not express any functional wild-type pIII any longer, and all antibody:pIII fusion proteins are displayed on the surfaces of recombinant phage. Therefore, an increase of display level is advantageous in that a greater number of particles will have at least one copy of the fusion and so are capable of participating in binding with target molecules during selection procedure. Access to diverse antibodies to a target antigen can be crucial to find the most useful candidates for the development of therapeutic antibody drugs. Furthermore, a phage display system in this invention can be applied in the development of polypeptide ligands that bind to specific target molecules with high affinity such as proteins or polypeptides, not to mention of antibody.

1. A mutant helper phage for packaging a phagemid vector containing filamentous virus genome of which at least a part of the gene of wild-type minor coat protein is deleted or defective, wherein conditional suppressive translation stop codon is introduced at the N-terminal of the gene of minor coat protein of the mutant helper phage.

2. The mutant helper phage of claim 1, wherein the filamentous virus is selected from the group consisting of fd, M13, f1, If1, Ike, Zj/Z, Ff, Xf, Pf1 and Pf3.

3. The mutant helper phage of claim 1 or claim 2, wherein the minor coat protein is pIII.

4. The mutant helper phage of any of claims 1 to 3, the conditional suppressive translation stop codon is selected from the group consisting of UAG (Amber), UAA (Ocher) and UGA (Opel) codons.

5. The mutant helper phage of any of claims 1 to 4, wherein the conditional suppressive translation stop codon is introduced by insertion or replacement of the codon at the N′-terminal of the minor coat protein gene.

6. The mutant helper phage of claim 5, wherein the replacement is performed by substituting Glu codon with amber codon.

7. The mutant helper phage of claim 1, wherein at least two conditional suppressive translation stop codons are introduced.

8. The mutant helper phage of claim 1, wherein the N′-terminal of the minor coat protein gene is the gene coding 90 amino acids including leader sequence of pIII.

9. The mutant helper phage of claim 1, wherein the mutant helper phage is selected from the group consisting of M13KO7, M13R408, M13-VCS and Phi X174.

10. The mutant helper phage of claim 1, wherein the mutant helper phage is M13KO7, the minor coat protein is pIII, and the 20^{th} and the 32^{th} Glu codons of the pIII N′-terminal are replaced with amber codons respectively (Deposit No.: KCTC 10022BP).

11. A method of preparing a recombinant virus library expressing genetically various foreign proteins on the surface thereof, comprising the steps of:
i) preparing a recombinant phagemid expressing active foreign protein as fused with anchor domain of pIII;
ii) co-infecting non-suppressive host cells with the phagemid of step i) and any of the mutant helper phages of claims 1 to 10; and
iii) incubating the co-infected host cells of the step ii), thereby obtaining recombinant virus expressing at least one of pIII-protein fusion protein.

12. The method of claim 11, wherein the phagemid includes filamentous virus genome which is selected from the group consisting of fd, M13, f1, If1, Ike, Zj/Z, Ff, Xf, Pf1 and Pf3.

13. The method of claim 11 or claim 12, wherein the active foreign protein of step i) is human immunoglobulin.

14. The method of claim 13, wherein the active foreign protein of step i) includes V_{H} domain and V_{L} domain of human immunoglobulin.

15. The method of claim 14, wherein the active foreign protein of step i) is scFv (single chain Fv) in which V_{H} domain is connected to V_{L} domain by a linker.

16. The method of any of claims 11-15, wherein the recombinant phagemid expresses fused protein including enterokinase and trypsin cleavage sites between pIII anchor domain and active foreign protein.

17. The method of claim 16, wherein the recombinant phagemid is pIGT3 which is shown in FIG. 3B (Deposit No.: KCTC 10021BP).

18. The method of any of claims 11-17, wherein the non-suppressive host-cell is selected from the group consisting of MV1184, MV1193, XS101, XS127 and JS5.

19. The method of any of claims 11-18, wherein the non-suppressive host cell is co-infected with bacteria having recombinant phagemid and mutant helper phages in ratio of 1:10 to 1:20.

20. The method of any of claims 11-19, wherein additional step of incubating suppressive host-cells infected with the mutant helper phages is included between step ii) and step iii).

21. The method of claim 20, wherein the suppressive host-cell is selected from the group consisting of M13KO7, M13R408, M13-VCS and Phi X174.

22. A method of selecting recombinant virus expressing antibodies binding to target antigens, which comprises affinity selection for the recombinant virus library generated by any of the methods of claims 11-20.

23. A method of isolating an antibody by treating the selected recombinant virus according to claim 22 with enterokinase or trypsin.
